SEQUENCE LISTING (9 sequences)

SEQ ID NO: 1 - protein, 8 aa, Artificial Sequence
  CDR-H1 of anti-CEACAM5-antibody
GFVFSSYD

SEQ ID NO: 2 - protein, 8 aa, Artificial Sequence
  CDR-H2 of anti-CEACAM5-antibody
ISSGGGIT

SEQ ID NO: 3 - protein, 13 aa, Artificial Sequence
  CDR-H3 of anti-CEACAM5-antibody
AAHYFGSSGPFAY

SEQ ID NO: 4 - protein, 6 aa, Artificial Sequence
  CDR-L1 of anti-CEACAM5-antibody
ENIFSY

SEQ ID NO: 5 - protein, 9 aa, Artificial Sequence
  CDR-L3 of anti-CEACAM5-antibody
QHHYGTPFT

SEQ ID NO: 6 - protein, 120 aa, Artificial Sequence [sequence omitted]

SEQ ID NO: 7 - protein, 107 aa, Artificial Sequence [sequence omitted]

SEQ ID NO: 8 - protein, 449 aa, Artificial Sequence
  Heavy chain of anti-CEACAM5-antibody
EVQLQESGPGLVKPGGSLSLSCAASGFVFSSYDMSWVRQTPERGLEWVAYISSGGGITYAPSTVKGRFTVSRDNAKNTLYLQMNSLTSEDTAVYYCAAHYFGSSGPFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 9 - protein, 214 aa, Artificial Sequence
  Light chain of anti-CEACAM5-antibody
DIQMTQSPASLSASVGDRVTITCRASENIFSYLAWYQQKPGKSPKLLVYNTRTLAEGVPSRFSGSGSGTDFSLTISSLQPEDFATYYCQHHYGTPFTFGSGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

(19) United States
(12) Patent Application Publication
(10) Pub. No.: US 2023/0149557 A1
(43) Pub. Date: May 18, 2023

(54) ANTITUMOR COMBINATIONS CONTAINING ANTI-CEACAM5 ANTIBODY CONJUGATES AND CETUXIMAB

(71) Applicant: SANOFI, Paris (FR)

(72) Inventor: Céline NICOLAZZI, Paris (FR)

(21) Appl. No.: 17/917,064

(22) PCT Filed: Apr. 22, 2021
(86) PCT No.: PCT/EP2021/060542

§ 371 Date: Oct. 5, 2022

(30) Foreign Application Priority Data
Apr. 24, 2020 (WO) 20315221.0

(51) Int. Cl. A61K 47/68; A61K 39/395; A61P 35/00

(52) U.S. Cl. CPC A61K 47/6853; A61K 47/6803; A61K 47/6889; A61K 39/3955; A61P 35/00; A61K 47/6805; A61K 47/6817; A61K 2039/545

(57) ABSTRACT

The present invention concerns antibody-conjugates comprising an anti-CEACAM5-antibody for use for treating cancer in combination with cetuximab. The invention further relates to pharmaceutical compositions and kit-of-parts comprising an anti-CEA-CAM5-antibody in combination with cetuximab for use for treating cancer.

### TECHNICAL BACKGROUND

The present invention concerns antibody-conjugates comprising an anti-CEACAM5-antibody for use for treating cancer in combination with cetuximab. The invention further relates to pharmaceutical compositions and kit-of-parts comprising an anti-CEACAM5-antibody in combination with cetuximab for use for treating cancer.

Carcino-embryonic antigen (CEA) is a glycoprotein involved in cell adhesion. CEA was first identified in 1965 (Gold and Freedman, J Exp Med, 121, 439, 1965) as a protein normally expressed by fetal gut during the first six months of gestation, and found in cancers of the pancreas, liver and colon. The CEA family belongs to the immunoglobulin superfamily. The CEA family, which consists of 18 genes, is sub-divided in two sub-groups of proteins: the carcinoembryonic antigen-related cell adhesion molecule (CEACAM) sub-group and the pregnancy-specific glycoprotein subgroup (Kammerer & Zimmermann, BMC Biology 2010, 8:12).

In humans, the CEACAM sub-group consists of 7 members: CEACAM1, CEACAM3, CEACAM4, CEACAM5, CEACAM6, CEACAM7, CEACAM8. Numerous studies have shown that CEACAM5, identical to the originally identified CEA, is highly expressed on the surface of colorectal, gastric, lung, breast, prostate, ovary, cervix, and bladder tumor cells and weakly expressed in few normal epithelial tissues such as columnar epithelial and goblet cells in colon, mucous neck cells in the stomach and squamous epithelial cells in esophagus and cervix (Hammarstrom et al, 2002, in “Tumor markers, Physiology, Pathobiology, Technology and Clinical Applications” Eds. Diamandis E. P. et al., AACC Press, Wash. pp 375). Thus, CEACAM5 may constitute a therapeutic target suitable for tumor specific targeting approaches, such as immunoconjugates.

The extracellular domains of CEACAM family members are composed of repeated immunoglobulin-like (Ig-like) domains which have been categorized in 3 types, A, B and N, according to sequence homologies. CEACAM5 contains seven such domains, namely N, A1, B1, A2, B2, A3 and B3. CEACAM5 A1, A2 and A3 domains, on one hand, and B1, B2 and B3 domains, on the other hand, show high sequence homologies, the A domains of human CEACAM5 presenting from 84 to 87% pairwise sequence similarity, and the B domains from 69 to 80%. Furthermore, other human CEACAM members presenting A or/and B domains in their structure, namely CEACAM1, CEACAM6, CEACAM7 and CEACAM8, show homology with human CEACAM5. In particular, the A and B domains of human CEACAM6 protein display sequence homologies with A1 and A3 domains, and any of B1 to B3 domains of human CEACAM5, respectively, which are even higher than observed among the A domains and the B domains of human CEACAM5.

Numerous anti-CEA antibodies were generated in view of CEA-targeted diagnostic or therapeutic purposes. Specificity towards related antigens has always been mentioned as a concern in this field, as an example by Sharkey et al (1990, Cancer Research 50, 2823). Due to the above mentioned homologies some of previously described antibodies may demonstrate binding to repetitive epitopes of CEACAM5 present in the different immunoglobulin domains and/or show cross-reactivity to other CEACAM members such as CEACAM1, CEACAM6, CEACAM7, or CEACAM8, lacking specificity to CEACAM5. The specificity of the anti-CEACAM5 antibody is desired in view of CEA-targeted therapies such that it binds to human CEACAM5-expressing tumor cells but does not bind to some normal tissues expressing the others CEACAM members. It is noteworthy that CEACAM1, CEACAM6 and CEACAM8 have been described as expressed by neutrophils of human and non-human primates (Ebrahimmnejad et al, 2000, Exp Cell Res, 260, 365; Zhao et al, 2004, J Immunol Methods 293, 207; Strickland et al, 2009 J Pathol, 218, 380) where they have been shown to regulate granulopoiesis and to play a role in immune response.

In the international patent application published as WO 2014/079886 is disclosed an antibody binding to the A3-B3 domain of human and Macaca fascicularis CEACAM5 proteins and which does not significantly cross-react with human CEACAM1, human CEACAM6, human CEACAM7, human CEACAM8, Macaca fascicularis CEACAM1, Macaca fascicularis CEACAM6, and Macaca fascicularis CEACAM8. This antibody has been conjugated to a maytansinoid, thereby providing the immunoconjugate having a significant cytotoxic activity on MKN45 human gastric cancer cells, with IC₅₀ values 1≤nM.

Antibody-immunoconjugates are comprised of an antibody attached to a cytostatic drug. According to an embodiment the cytostatic drug is attached to the cytostatic drug via a chemical linker. These immunoconjugates have great potential in cancer chemotherapy and enable selective delivery of a potent cytostatic to target cancer cells, resulting in improved efficacy, reduced systemic toxicity, and improved pharmacokinetics, pharmacodynamics and biodistribution compared to traditional chemotherapy. To date, hundreds of diverse immunoconjugates have been developed against various cancers, of which several have been approved for human use.

The majority of chemotherapy regimens nowadays aim at the administration of a combination of cytotoxic drugs, each drug with a different mechanism of action and favorably with synergistic effects, causing the death of cancer cells. Such a chemotherapy regimen is typically defined by the cytotoxic drugs used, their dosage, administration frequency and duration. Over the decades, new chemotherapy regimens have been developed and existing chemotherapy regimens have been refined for the treatment of cancers.

However, according to the World Health Organization, cancer was the second leading cause of death globally and responsible for approx. 9.6 million in 2018. Thus, there is continued need for providing improved drug combinations and regimens for the treatment of cancer.

### SUMMARY OF THE INVENTION

The present invention relates to an immunoconjugate comprising an anti-CEACAM5-antibody which is for use in combination with cetuximab for the treatment of cancer.

The present invention further relates to a pharmaceutical composition comprising the immunoconjugate comprising an anti-CEACAM5-antibody and cetuximab, and further the use of the pharmaceutical composition for the treatment of cancer.

The present invention also relates a kit comprising (i) a pharmaceutical composition comprising an immunoconjugate comprising an anti-CEACAM5-antibody and (ii) a pharmaceutical composition comprising cetuximab, in separate or combined formulations.

The invention and further relates to the use of the kit for the treatment of cancer.

While by far not all possible combinations of cytostatic agents show a further improved therapeutic effect, the present inventors have determined that specifically the immunoconjugate comprising an anti-CEACAM5-antibody in combination with cetuximab shows favorable activity for the treatment of cancer relative to the administration of anti-CEACAM5-antibody or cetuximab alone.

### DETAILED DESCRIPTION OF THE INVENTION

### Definitions

An “antibody” may be a natural or conventional antibody in which two heavy chains are linked to each other by disulfide bonds and each heavy chain is linked to a light chain by a disulfide bond. There are two types of light chain, lambda (I) and kappa (k). There are five main heavy chain classes (or isotypes) which determine the functional activity of an antibody molecule: IgM, IgD, IgG, IgA and IgE. Each chain contains distinct sequence domains. The light chain includes two domains or regions, a variable domain (VL) and a constant domain (CL). The heavy chain includes four domains, a variable domain (VH) and three constant domains (CH1, CH2 and CH3, collectively referred to as CH). The variable regions of both light (VL) and heavy (VH) chains determine binding recognition and specificity to the antigen. The constant region domains of the light (CL) and heavy (CH) chains confer important biological properties, such as antibody chain association, secretion, trans-placental mobility, complement binding, and binding to Fc receptors (FcR). The Fv fragment is the N-terminal part of the Fab fragment of an immunoglobulin and consists of the variable portions of one light chain and one heavy chain. The specificity of the antibody resides in the structural complementarity between the antibody combining site and the antigenic determinant. Antibody combining sites are made up of residues that are primarily from the hypervariable or complementarity determining regions (CDRs). Occasionally, residues from non-hypervariable or framework regions (FR) influence the overall domain structure and hence the combining site. Complementarity Determining Regions or CDRs therefore refer to amino acid sequences which together define the binding affinity and specificity of the natural Fv region of a native immunoglobulin binding site. The light and heavy chains of an immunoglobulin each have three CDRs, designated CDR1-L, CDR2-L, CDR3-L and CDR1-H, CDR2-H, CDR3-H, respectively. A conventional antibody antigen-binding site, therefore, includes six CDRs, comprising the CDR set from each of a heavy and a light chain V region.

“Framework Regions” (FRs) refer to amino acid sequences interposed between CDRs, i.e. to those portions of immunoglobulin light and heavy chain variable regions that are relatively conserved among different immunoglobulins in a single species. The light and heavy chains of an immunoglobulin each have four FRs, designated FR1-L, FR2-L, FR3-L, FR4-L, and FR1-H, FR2-H, FR3-H, FR4-H, respectively. A human framework region is a framework region that is substantially identical (about 85%, or more, in particular 90%, 95%, 97%, 99% or 100%) to the framework region of a naturally occurring human antibody.

In the context of the invention, CDR/FR definition in an immunoglobulin light or heavy chain is to be determined based on IMGT definition (Lefranc et al. Dev. Comp. Immunol., 2003, 27(1):55-77; www.imgt.org).

As used herein, the term “antibody” denotes conventional antibodies and fragments thereof, as well as single domain antibodies and fragments thereof, in particular variable heavy chain of single domain antibodies, and chimeric, humanised, bispecific or multispecific antibodies.

As used herein, antibody or immunoglobulin also includes “single domain antibodies” which have been more recently described and which are antibodies whose complementary determining regions are part of a single domain polypeptide. Examples of single domain antibodies include heavy chain antibodies, antibodies naturally devoid of light chains, single domain antibodies derived from conventional four-chain antibodies, engineered single domain antibodies. Single domain antibodies may be derived from any species including, but not limited to mouse, human, camel, llama, goat, rabbit, bovine.

Single domain antibodies may be naturally occurring single domain antibodies known as heavy chain antibody devoid of light chains. In particular, camelidae species, for example camel, dromedary, llama, alpaca and guanaco, produce heavy chain antibodies naturally devoid of light chain. Camelid heavy chain antibodies also lack the CH1 domain.

The variable heavy chain of these single domain antibodies devoid of light chains are known in the art as “VHH” or “nanobodv”. Similar to conventional VH domains, VHHs contain four FRs and three CDRs. Nanobodies have advantages over conventional antibodies: they are about ten times smaller than IgG molecules, and as a consequence properly folded functional nanobodies can be produced by in vitro expression while achieving high yield. Furthermore, nanobodies are very stable, and resistant to the action of proteases. The properties and production of nanobodies have been reviewed by Harmsen and De Haard HJ (Appl. Microbiol. Biotechnol. 2007 November; 77(1):13-22).

The term “monoclonal antibody” or “mAb” as used herein refers to an antibody molecule of a single amino acid sequence, which is directed against a specific antigen, and is not to be construed as requiring production of the antibody by any particular method. A monoclonal antibody may be produced by a single clone of B cells or hybridoma, but may also be recombinant, i.e. produced by protein engineering.

The term “humanised antibody” refers to an antibody which is wholly or partially of non-human origin and which has been modified to replace certain amino acids, in particular in the framework regions of the VH and VL domains, in order to avoid or minimize an immune response in humans. The constant domains of a humanized antibody are most of the time human CH and CL domains.

“Fragments” of (conventional) antibodies comprise a portion of an intact antibody, in particular the antigen binding region or variable region of the intact antibody. Examples of antibody fragments include Fv, Fab, F(ab′)2, Fab′, dsFv, (dsFv)2, scFv, sc(Fv)2, diabodies, bispecific and multispecific antibodies formed from antibody fragments. A fragment of a conventional antibody may also be a single domain antibody, such as a heavy chain antibody or VHH.

The term “Fab” denotes an antibody fragment having a molecular weight of about 50,000 and antigen binding activity, in which about a half of the N-terminal side of the heavy chain and the entire light chain are bound together through a disulfide bond. It is usually obtained among fragments by treating IgG with a protease, such as papaine.

The term “F(ab′)2” refers to an antibody fragment having a molecular weight of about 100,000 and antigen binding activity, which is slightly larger than 2 identical Fab fragments bound via a disulfide bond of the hinge region. It is usually obtained among fragments by treating IgG with a protease, such as pepsin.

The term “Fab” refers to an antibody fragment having a molecular weight of about 50,000 and antigen binding activity, which is obtained by cutting a disulfide bond of the hinge region of the F(ab′)2.

A single chain Fv (“scFv”) polypeptide is a covalently linked VH::VL heterodimer which is usually expressed from a gene fusion including VH and VL encoding genes linked by a peptide-encoding linker. The human scFv fragment of the invention includes CDRs that are held in appropriate conformation, in particular by using gene recombination techniques. Divalent and multivalent antibody fragments can form either spontaneously by association of monovalent scFvs, or can be generated by coupling monovalent scFvs by a peptide linker, such as divalent sc(Fv)2. “dsFv” is a VH::VL heterodimer stabilised by a disulphide bond. “(dsFv)2” denotes two dsFv coupled by a peptide linker.

The term “bispecific antibody” or “BsAb” denotes an antibody which combines the antigen-binding sites of two antibodies within a single molecule. Thus, BsAbs are able to bind two different antigens simultaneously. Genetic engineering has been used with increasing frequency to design, modify, and produce antibodies or antibody derivatives with a desired set of binding properties and effector functions as described for instance in EP 2 050 764 A1.

The term “multispecific antibody” denotes an antibody which combines the antigen-binding sites of two or more antibodies within a single molecule.

The term “diabodies” refers to small antibody fragments with two antigen-binding sites, which fragments comprise a heavy-chain variable domain (VH) connected to a light-chain variable domain (VL) in the same polypeptide chain (VH-VL). By using a linker that is too short to allow pairing between the two domains of the same chain, the domains are forced to pair with the complementary domains of another chain and create two antigen-binding sites.

An amino acid sequence “at least 85% identical to a reference sequence” is a sequence having, on its entire length, 85%, or more, in particular 90%, 91%, 92%, 93%, 94%, 95%, 96%, 97%, 98% or 99% sequence identity with the entire length of the reference amino acid sequence.

A percentage of “sequence identity” between amino acid sequences may be determined by comparing the two sequences, optimally aligned over a comparison window, wherein the portion of the polynucleotide or polypeptide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity. Optimal alignment of sequences for comparison is conducted by global pairwise alignment, e.g. using the algorithm of Needleman and Wunsch J. Mol. Biol. 48:443 (1970). The percentage of sequence identity can be readily determined for instance using the program Needle, with the BLOSUM62 matrix, and the following parameters gap-open=10, gap-extend=0.5.

A “conservative amino acid substitution” is one in which an amino acid residue is substituted by another amino acid residue having a side chain R group with similar chemical properties (e.g., charge, size or hydrophobicity). In general, a conservative amino acid substitution will not substantially change the functional properties of a protein. Examples of groups of amino acids that have side chains with similar chemical properties include 1) aliphatic side chains: glycine, alanine, valine, leucine, and isoleucine; 2) aliphatic-hydroxyl side chains: serine and threonine; 3) amide-containing side chains: asparagine and glutamine; 4) aromatic side chains: phenylalanine, tyrosine, and tryptophan; 5) basic side chains: lysine, arginine, and histidine; 6) acidic side chains: aspartic acid and glutamic acid; and 7) sulfur-containing side chains: cysteine and methionine. Conservative amino acids substitution groups can also be defined on the basis of amino acid size.

By “purified” and “isolated” it is meant, when referring to a polypeptide (i.e. the antibody of the invention) or a nucleotide sequence, that the indicated molecule is present in the substantial absence of other biological macromolecules of the same type. The term “purified” as used herein in particular means at least 75%, 85%, 95%, or 98% by weight, of biological macromolecules of the same type are present. An “isolated” nucleic acid molecule which encodes a particular polypeptide refers to a nucleic acid molecule which is substantially free of other nucleic acid molecules that do not encode the subject polypeptide;

however, the molecule may include some additional bases or moieties which do not deleteriously affect the basic characteristics of the composition.

As used herein, the term “subject” denotes a mammal, such as a rodent, a feline, a canine, and a primate. In particular, a subject according to the invention is a human.

### Immunoconjugate Comprising an Anti-CEACAM5-Antibody

The present invention relates to an immunoconjugate comprising an anti-CEACAM5-antibody which is used in combination with cetuximab for the treatment of cancer.

The immunoconjugate typically comprises an anti-CEACAM5-antibody and at least one cytostatic agent. In particular, in the immunoconjugate, the anti-CEACAM5-antibody is covalently attached via a cleavable or non-cleavable linker to the at least one cytostatic agent.

### Anti-CEACAM5-Anti Body

According to an embodiment, the immunoconjugate comprises a humanized anti-CEACAM5-antibody.

According to an embodiment, the immunoconjugate comprises an anti-CEACAM5-antibody, wherein the anti-CEACAM5-antibody comprises a CDR-H1 consisting of SEQ ID NO: 1, CDR-H2 consisting of SEQ ID NO: 2, CDR-H3 consisting of SEQ ID NO: 3, CDR-L1 consisting of SEQ ID NO: 4, CDR-L2 consisting of amino acid sequence NTR, and CDR-L3 consisting of SEQ ID NO: 5.

In a further embodiment, the immunoconjugate comprises an anti-CEACAM5-antibody, wherein the anti-CEACAM5-antibody comprises a variable domain of a heavy chain (VH) consisting of SEQ ID NO: 6 and a variable domain of a light chain (VL) consisting of SEQ ID NO: 7.

The immunoconjugate comprises in a further embodiment an anti-CEACAM5-antibody, which comprises:
a variable domain of heavy chain consisting of sequence EVQLQESGPGLVKPGGSLSL SCAASGFVFSSYDMSWVRQTPERGLEWVAYISSGGGITYAPSTVKGRFTVSRDNAKNTL YLQMNSLTSEDTAVYYCAAHYFGSSGPFAYWGQGTLVTVSS (SEQ ID NO: 6, with CDRs shown in bold characters) in which FR1-H spans amino acid positions 1 to 25, CDR1-H spans amino acid positions 26 to 33 (SEQ ID NO: 1), FR2-H spans amino acid positions 34 to 50, CDR2-H spans amino acid positions 51 to 58 (SEQ ID NO: 2), FR3-H spans amino acid positions 59 to 96, CDR3-H spans amino acid positions 97 to 109 (SEQ ID NO: 3), and FR4-H spans amino acid positions 110 to 120, and
a variable domain of light chain consisting of sequence DIQMTQSPASLSASVGDRVTITCRASENIFSYLAWYQQKPGKSPKLLVYNTRTLAEGVPS FSGSGSGTDFSLTISSLQPEDFATYYCQHHYGTPFTFGSGTKLEIK (SEQ ID NO: 7, with CDRs shown in bold characters) in which FR1-L spans amino acid positions 1 to 26, CDR1-L spans amino acid positions 27 to 32 (SEQ ID NO: 4), FR2-L spans amino acid positions 33 to 49, CDR2-L spans amino acid positions 50 to 52, FR3-L spans amino acid positions 53 to 88, CDR3-L spans amino acid positions 89 to 97 (SEQ ID NO: 5), and FR4-L spans amino acid positions 98 to 107.

In a further embodiment, the immunoconjugate also comprises an anti-CEACAM5-antibody, wherein the anti-CEACAM5-antibody comprises a variable domain of a heavy chain (VH) having at least 90% identity to SEQ ID NO: 6, and a variable domain of a light chain (VL) having at least 90% identity to SEQ ID NO: 7, wherein CDR1-H consists of SEQ ID NO: 2, CDR2-H consists of SEQ ID NO: 3, CDR3-H consists of SEQ ID NO: 4, CDR1-L consists of SEQ ID NO: 6, CDR2-L consists of amino acid sequence NTR, and CDR3-L consists of SEQ ID NO: 7.

In a further embodiment, the immunoconjugate comprises an anti-CEACAM5-antibody, wherein the anti-CEACAM5-antibody comprises a variable domain of a heavy chain (VH) having at least 92%, at least 95%, at least 98% identity to SEQ ID NO: 6, and a variable domain of a light chain (VL) having at least 92%, at least 95%, at least 98% identity to SEQ ID NO: 7, wherein CDR1-H consists of SEQ ID NO: 2, CDR2-H consists of SEQ ID NO: 3, CDR3-H consists of SEQ ID NO: 4, CDR1-L consists of SEQ ID NO: 6, CDR2-L consists of amino acid sequence NTR, and CDR3-L consists of SEQ ID NO: 7.

In a further embodiment, the immunoconjugate comprises an anti-CEACAM5-antibody, wherein the anti-CEACAM5-antibody comprises a heavy chain (VH) consisting of SEQ ID NO: 8 and a light chain (VL) consisting of SEQ ID NO: 9.

In a further embodiment, the immunoconjugate comprises an anti-CEACAM5-antibody, wherein the anti-CEACAM5-antibody comprises a heavy chain (VH) having at least 90% sequence identity to SEQ ID NO: 8 and a light chain (VL) having at least 90% sequence identity to SEQ ID NO: 9, wherein CDR1-H consists of SEQ ID NO: 2, CDR2-H consists of SEQ ID NO: 3, CDR3-H consists of SEQ ID NO: 4, CDR1-L consists of SEQ ID NO: 6, CDR2-L consists of amino acid sequence NTR, and CDR3-L consists of SEQ ID NO: 7.

In a further embodiment, the immunoconjugate comprises an anti-CEACAM5-antibody, wherein the anti-CEACAM5-antibody comprises a heavy chain (VH) having at least 92%, at least 95%, at least 98% identity to SEQ ID NO: 8 and a light chain (VL) having at least 92%, at least 95%, at least 98% identity to SEQ ID NO: 9, wherein CDR1-H consists of SEQ ID NO: 2, CDR2-H consists of SEQ ID NO: 3, CDR3-H consists of SEQ ID NO: 4, CDR1-L consists of SEQ ID NO: 6, CDR2-L consists of amino acid sequence NTR, and CDR3-L consists of SEQ ID NO: 7.

The anti-CEACAM5-antibody comprised in the immunoconjugate may also be a single domain antibody or a fragment thereof. In particular, a single domain antibody fragment may consist of a variable heavy chain (VHH) which comprises the CDR1-H, CDR2-H and CDR3-H of the antibodies as described above. The antibody may also be a heavy chain antibody, i.e. an antibody devoid of light chain, which may or may not contain a CH1 domain.

The single domain antibody or a fragment thereof may also comprise the framework regions of a camelid single domain antibody, and optionally the constant domain of a camelid single domain antibody.

The anti-CEACAM5-antibody comprised in the immunoconjugate may also be an antibody fragment, in particular a humanised antibody fragment, selected from the group consisting of Fv, Fab, F(ab′)2, Fab′, dsFv, (dsFv)2, scFv, sc(Fv)2, and diabodies.

The antibody may also be a bispecific or multispecific antibody formed from antibody fragments, at least one antibody fragment being an antibody fragment according to the invention. Multispecific antibodies are polyvalent protein complexes as described for instance in EP 2 050 764 A1 or US 2005/0003403 A1.

The anti-CEACAM5-antibody and fragments thereof comprised in the immunoconjugate can be produced by any technique well known in the art. In particular said antibodies are produced by techniques as hereinafter described.

The anti-CEACAM5-antibody and fragments thereof comprised in the immunoconjugate can be used in an isolated (e.g., purified) from or contained in a vector, such as a membrane or lipid vesicle (e.g. a liposome).

The anti-CEACAM5-antibody and fragments thereof comprised in the immunoconjugate may be produced by any technique known in the art, such as, without limitation, any chemical, biological, genetic or enzymatic technique, either alone or in combination.

Knowing the amino acid sequence of the desired sequence, one skilled in the art can readily produce anti-CEACAM5-antibody and fragments thereof, by standard techniques for production of polypeptides. For instance, they can be synthesized using well-known solid phase method, in particular using a commercially available peptide synthesis apparatus (such as that made by Applied Biosystems, Foster City, Calif.) and following the manufacturer's instructions. Alternatively, anti-CEACAM5-antibody and fragments thereof can be synthesized by recombinant DNA techniques as is well-known in the art. For example, these fragments can be obtained as DNA expression products after incorporation of DNA sequences encoding the desired (poly)peptide into expression vectors and introduction of such vectors into suitable eukaryotic or prokaryotic hosts that will express the desired polypeptide, from which they can be later isolated using well-known techniques.

Anti-CEACAM5-antibody and fragments thereof are suitably separated from the culture medium by conventional immunoglobulin purification procedures such as, for example, protein A-Sepharose, hydroxylapatite chromatography, gel electrophoresis, dialysis, or affinity chromatography.

Methods for producing humanised antibodies based on conventional recombinant DNA and gene transfection techniques are well known in the art (See, e.g., Riechmann L. et al. 1988; Neuberger MS. et al. 1985). Antibodies can be humanised using a variety of techniques known in the art including, for example, the technique disclosed in the application WO2009/032661, CDR-grafting (EP 239,400; PCT publication WO91/09967; U.S. Pat. Nos. 5,225,539; 5,530,101; and 5,585,089), veneering or resurfacing (EP 592,106; EP 519,596; Padlan EA (1991); Studnicka GM et al. (1994); Roguska MA. et al. (1994)), and chain shuffling (U.S. Pat. No. 5,565,332). The general recombinant DNA technology for preparation of such antibodies is also known (see European Patent Application EP 125023 and International Patent Application WO 96/02576).

The Fab of the anti-CEACAM5-antibody can be obtained by treating an antibody which specifically reacts with CEACAM5 with a protease, such as papaine. Also, the Fab of the anti-CEACAM5-antibody can be produced by inserting DNA sequences encoding both chains of the Fab of the anti-CEACAM5-antibody into a vector for prokaryotic expression, or for eukaryotic expression, and introducing the vector into prokaryotic or eukaryotic cells (as appropriate) to express the Fab of the anti-CEACAM5-antibody.

The F(ab′)2 of the anti-CEACAM5-antibody can be obtained treating an antibody which specifically reacts with CEACAM5 with a protease, such as pepsin. Also, the F(ab′)2 of the anti-CEACAM5-antibody can be produced by binding Fab′ described below via a thioether bond or a disulfide bond.

The Fab′ of the of the anti-CEACAM5-antibody can be obtained treating F(ab′)2 which specifically reacts with CEACAM5 with a reducing agent, such as dithiothreitol. Also, the Fab′ of the anti-CEACAM5-antibody can be produced by inserting DNA sequences encoding Fab′ chains of the antibody into a vector for prokaryotic expression, or a vector for eukaryotic expression, and introducing the vector into prokaryotic or eukaryotic cells (as appropriate) to perform its expression.

The scFv of the of the anti-CEACAM5-antibody can be produced by taking sequences of the CDRs or VH and VL domains as previously described, constructing a DNA encoding an scFv fragment, inserting the DNA into a prokaryotic or eukaryotic expression vector, and then introducing the expression vector into prokaryotic or eukaryotic cells (as appropriate) to express the scFv. To generate a humanised scFv fragment, a well-known technology called CDR grafting may be used, which involves selecting the complementary determining regions (CDRs) according to the invention, and grafting them onto a human scFv fragment framework of known three dimensional structure (see, e.g., WO98/45322; WO 87/02671; U.S. Pat. Nos. 5,859,205; 5,585,089; 4,816,567; EP0173494).

### Cytostatic Agents

The immunoconjugate for the use according to the present invention typically comprises at least one cytostatic agent. A cytostatic agent as used herein refers to an agent that kills cells, including cancer cells. Such agents favorably stop cancer cells from dividing and growing and cause tumors to shrink in size. The term cytostatic agent is used herein interchangeably with the terms chemotherapeutic agent, cytotoxic agent, or cytostatic.

In a further embodiment, the cytostatic agent is selected from the group consisting of radioisotopes, protein toxins, small molecule toxins, and combinations thereof.

Radioisotopes include radioactive isotopes suitable for treating cancer. Such radioisotopes generally emit mainly beta-radiation. In a further embodiment, the radioisotopes are selected from the group consisting of At²¹¹, Bi²¹², Er¹⁶⁹, I¹³¹, I¹²⁵, Y⁹⁰, In¹¹¹, P³², Re¹⁸⁶, Re¹⁸⁸, Sm¹⁵³, Sr⁸⁹, radioactive isotopes of Lu, and combinations thereof. In an embodiment, the radioactive isotope is alpha-emitter isotope, more specifically Th²²⁷, which emits alpha-radiation.

In a further embodiment, the small molecule toxins are selected from antimetabolites, DNA-alkylating agents, DNA-cross-linking agents, DNA-intercalating agents, anti-microtubule agents, topoisomerase inhibitors, and combinations thereof.

In a further embodiment, the anti-microtubule agent is selected from the group consisting of taxanes, vinca alkaloids, maytansinoids, colchicine, podophyllotoxin, gruseofulvin, and combinations thereof.

According to an embodiment, maytansinoids are selected from maytansinol, maytansinol analogs, and combinations thereof.

Examples of suitable maytansinol analogues include those having a modified aromatic ring and those having modifications at other positions. Such suitable maytansinoids are disclosed in U.S. Pat. Nos. 4,424,219; 4,256,746; 4,294,757; 4,307,016; 4,313,946; 4,315,929; 4,331,598; 4,361,650; 4,362,663; 4,364,866; 4,450,254; 4,322,348; 4,371,533; 6,333,410; 5,475,092; 5,585,499; and 5,846,545.

Specific examples of suitable analogues of maytansinol having a modified aromatic ring include:

(1) C-19-dechloro (U.S. Pat. No. 4,256,746) (prepared by LAH reduction of ansamytocin P2);

(2) C-20-hydroxy (or C-20-demethyl)+/−C-19-dechloro (U.S. Pat. Nos. 4,361,650 and 4,307,016) (prepared by demethylation using Streptomyces or Actinomyces or dechlorination using LAH); and

(3) C-20-demethoxy, C-20-acyloxy (—OCOR), +/− dechloro (U.S. Pat. No. 4,294,757) (prepared by acylation using acyl chlorides).

Specific examples of suitable analogues of maytansinol having modifications of other positions include:

(1) C-9-SH (U.S. Pat. No. 4,424,219) (prepared by the reaction of maytansinol with H2S or P2S5);

(2) C-14-alkoxymethyl (demethoxy/CH2OR) (U.S. Pat. No. 4,331,598);

(3) C-14-hydroxymethyl or acyloxymethyl (CH2OH or CH2OAc) (U.S. Pat. No. 4,450,254) (prepared from Nocardia);

(4) C-15-hydroxy/acyloxy (U.S. Pat. No. 4,364,866) (prepared by the conversion of maytansinol by Streptomyces);

(5) C-15-methoxy (U.S. Pat. Nos. 4,313,946 and 4,315,929) (isolated from Trewia nudiflora);

(6) C-18-N-demethyl (U.S. Pat. Nos. 4,362,663 and 4,322,348) (prepared by the demethylation of maytansinol by Streptomyces); and

(7) 4,5-deoxy (U.S. Pat. No. 4,371,533) (prepared by the titanium trichloride/LAH reduction of maytansinol).

In a further embodiment, the cytotoxic conjugates of the present invention utilize the thiol-containing maytansinoid (DM1), formally termed N2′-deacetyl-N2′-(3-mercapto-1-oxopropyl)-maytansine, as the cytotoxic agent. DM1 is represented by the following structural formula (I):

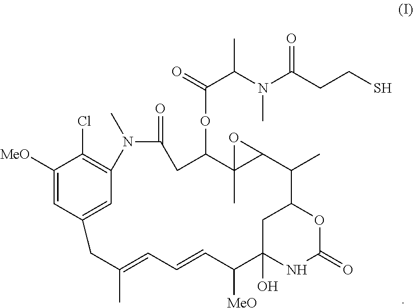 [SMILES: COc1cc2cc(c1Cl)N(C)C(=O)CC(OC(=O)C(C)N(C)C(=O)CCS)C1(C)OC1C(C)C1CC(O)(NC(=O)O1)C(OC)/C=C/C=C(\C)C2]

In a further embodiment, the cytotoxic conjugates of the present invention utilize the thiol-containing maytansinoid DM4, formally termed N2′-deacetyl-N-2′(4-methyl mercapto-1-oxopentyl)-maytansine, as the cytotoxic agent. DM4 is represented by the following structural formula (II):

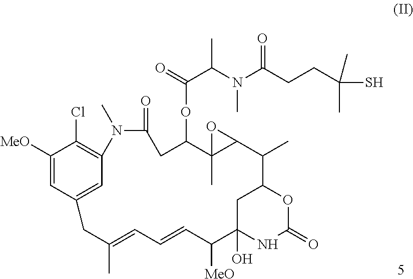 [SMILES: COc1cc2cc(c1Cl)N(C)C(=O)CC(OC(=O)C(C)N(C)C(=O)CCC(C)(C)S)C1(C)OC1C(C)C1CC(O)(NC(=O)O1)C(OC)/C=C/C=C(\C)C2]

In further embodiments of the invention, other maytansines, including thiol and disulfide-containing maytansinoids bearing a mono or di-alkyl substitution on the carbon atom bearing the sulfur atom, may be used. These include a maytansinoid having, at C-3, C-14 hydroxymethyl, C-15 hydroxy, or C-20 desmethyl, an acylated amino acid side chain with an acyl group bearing a hindered sulfhydryl group, wherein the carbon atom of the acyl group bearing the thiol functionality has one or two substituents, said substituents being CH3, C2H5, linear or branched alkyl or alkenyl having from 1 to 10 reagents and any aggregate which may be present in the solution.

Accordingly, in a further embodiment, the maytansinoids are selected from the group consisting of (N2′-deacetyl-N2′-(3-mercapto-1-oxopropyl)-maytansine) DM1 or N2′-deacetyl-N-2′(4-methyl-4-mercapto-1-oxopentyl)-maytansine (DM4), and combinations thereof.

In a further embodiment, in the immunoconjugate, the anti-CEACAM5-antibody is covalently attached via a cleavable or non-cleavable linker to the at least one cytostatic agent.

In a further embodiment, the linker is selected from the group consisting of N-succinimidyl pyridyldithiobutyrate (SPDB), 4-(pyridin-2-yldisulfanyl)-2-sulfo-butyric acid (sulfo-SPDB), and succinimidyl(N-maleimidomethyl) cyclohexane-1-carboxylate (SMCC).

In a further embodiment, the linker binds to a lysine residue in the Fc region of the anti-CEACAM5 antibody. In a further embodiment, the linker forms a disulfide bond or a thioether bond with the maytansine.

In particular, the anti-CEACAM5-immunoconjugate may be selected from the group consisting of:

i) the anti-CEACAM5-SPDB-DM4-immunoconjugate of formula (III)

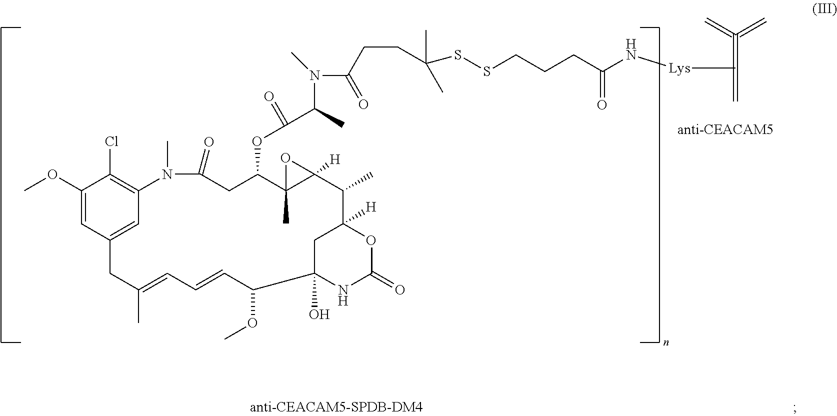

ii) anti-CEACAM5-sulfo-SPDB-DM4-immunoconjugate of formula (IV)

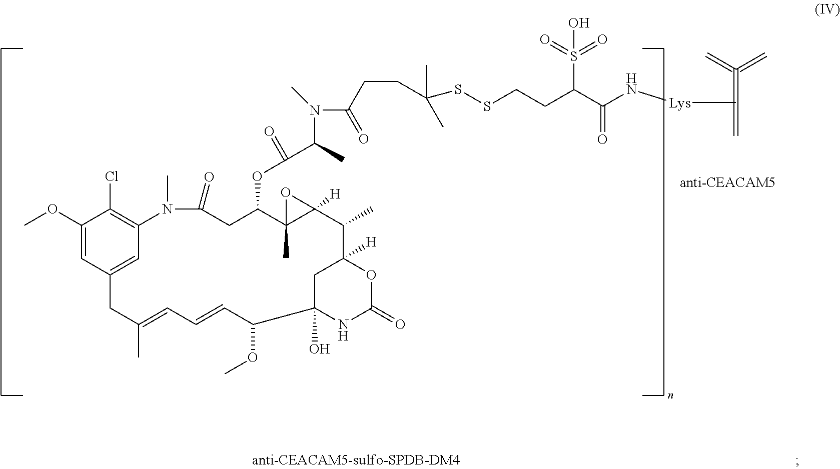

and

iii) anti-CEACAM5-SMCC-DM1-immunoconjugate of formula (V)

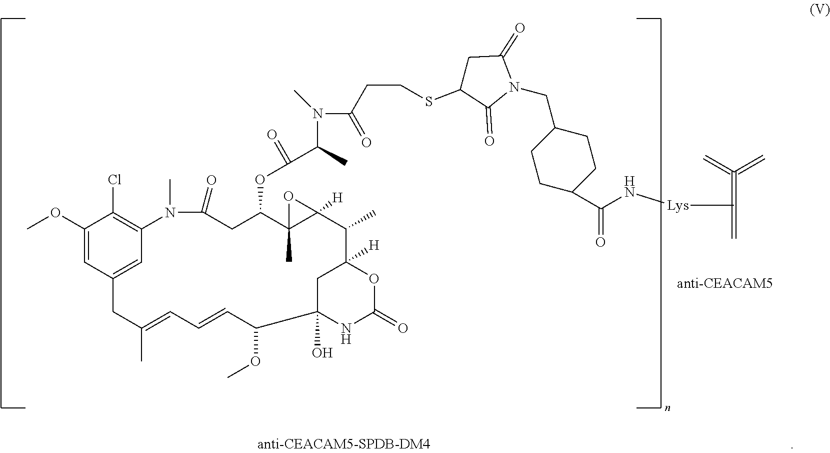

In a further embodiment, the immunoconjugate of the present invention comprises an anti-CEACAM5-antibody, which comprises a heavy chain (VH) of SEQ ID NO: 8 and a light chain (VL) of SEQ ID NO: 9 (huMAb2-3), wherein huMAb2-3 is covalently linked to N2′-deacetyl-N-2′(4-methyl-4-mercapto-1-oxopentyl)-maytansine (DM4) via N-succinimidyl pyridyldithiobutyrate (SPDB). Thereby, the immunoconjugate huMAb2-3-SPDB-DM4 is obtained.

“Linker”, as used herein, means a chemical moiety comprising a covalent bond or a chain of atoms that covalently attaches a polypeptide to a drug moiety.

The conjugates may be prepared by in vitro methods. In order to link a drug or prodrug to the antibody, a linking group is used. Suitable linking groups are well known in the art and include disulfide groups, thioether groups, acid labile groups, photolabile groups, peptidase labile groups and esterase labile groups. Conjugation of an antibody of the invention with cytotoxic agents or growth inhibitory agents may be made using a variety of bifunctional protein coupling agents including but not limited to N-succinimidyl pyridyldithiobutyrate (SPDB), butanoic acid 4-[(5-nitro-2-pyridinyl)dithio]-2,5-dioxo pyrrolidinyl ester (nitro-SPDB), 4-(pyridin-2-yldisulfanyl)-2-sulfo-butyric acid (sulfo-SPDB), N-succinimidyl (2-pyridyldithio) propionate (SPDP), succinimidyl (N-maleimidomethyl) cyclohexane-1-carboxylate (SMCC), iminothiolane (IT), bifunctional derivatives of imidoesters (such as dimethyl adipimidate HCL), active esters (such as disuccinimidyl suberate), aldehydes (such as glutaraldehyde), bis-azido compounds (such as bis (p-azidobenzoyl)-hexanediamine), bis-diazonium derivatives (such as bis-(p-diazoniumbenzoyl)-ethylenediamine), diisocyanates (such as toluene 2,6-diisocyanate), and bis-active fluorine compounds (such as 1,5-difluoro-2,4-dinitrobenzene). For example, a ricin immunotoxin can be prepared as described in Vitetta et al (1987). Carbon labeled 1-isothiocyanatobenzyl methyldiethylene triaminepentaacetic acid (MX-DTPA) is an exemplary chelating agent for conjugation of radionucleotide to the antibody (WO 94/11026).

The linker may be a “cleavable linker” facilitating release of the cytotoxic agent or growth inhibitory agent in the cell. For example, an acid-labile linker, a peptidase-sensitive linker, an esterase labile linker, a photolabile linker or a disulfide-containing linker (See e.g. U.S. Pat. No. 5,208,020) may be used. The linker may be also a “non-cleavable linker” (for example SMCC linker) that might led to better tolerance in some cases.

In general, the conjugate can be obtained by a process comprising the steps of:

(i) bringing into contact an optionally-buffered aqueous solution of a cell-binding agent (e.g. an antibody according to the invention) with solutions of a linker and a cytotoxic compound;

(ii) then optionally separating the conjugate which was formed in (i) from the unreacted cell-binding agent.

The aqueous solution of cell-binding agent can be buffered with buffers such as, e.g. potassium phosphate, acetate, citrate or N-2-Hydroxyethylpiperazine-N′-2-ethanesulfonic acid (Hepes buffer). The buffer depends upon the nature of the cell-binding agent. The cytotoxic compound is in solution in an organic polar solvent, e.g. dimethyl sulfoxide (DMSO) or dimethylacetamide (DMA).

The reaction temperature is usually comprised between 20 and 40° C. The reaction time can vary from 1 to 24 hours. The reaction between the cell-binding agent and the cytotoxic agent can be monitored by size exclusion chromatography (SEC) with a refractometric and/or UV detector. If the conjugate yield is too low, the reaction time can be extended.

A number of different chromatography methods can be used by the person skilled in the art in order to perform the separation of step (ii): the conjugate can be purified e.g. by SEC, adsorption chromatography (such as ion exchange chromatography, IEC), hydrophobic interaction chromatography (HIC), affinity chromatography, mixed-support chromatography such as hydroxyapatite chromatography, or high performance liquid chromatography (HPLC). Purification by dialysis or diafiltration can also be used.

As used herein, the term “aggregates” means the associations which can be formed between two or more cell-binding agents, said agents being modified or not by conjugation. The aggregates can be formed under the influence of a great number of parameters, such as a high concentration of cell-binding agent in the solution, the pH of the solution, high shearing forces, the number of bonded dimers and their hydrophobic character, the temperature (see Wang & Gosh, 2008, J. Membrane Sci., 318: 311-316, and references cited therein); note that the relative influence of some of these parameters is not clearly established. In the case of proteins and antibodies, the person skilled in the art will refer to Cromwell et al. (2006, AAPS Jounal, 8(3): E572-E579). The content in aggregates can be determined with techniques well known to the skilled person, such as SEC (see Walter et al., 1993, Anal. Biochem., 212(2): 469-480).

After step (i) or (ii), the conjugate-containing solution can be submitted to an additional step (iii) of chromatography, ultrafiltration and/or diafiltration.

The conjugate is recovered at the end of these steps in an aqueous solution.

In a further embodiment, the immunoconjugate according to the invention is characterised by a “drug-to-antibody ratio” (or “DAR”) ranging from 1 to 10, or from 2 to 5, or from 3 to 4. This is generally the case of conjugates including maytansinoid molecules.

This DAR number can vary with the nature of the antibody and of the drug (i.e. the growth-inhibitory agent) used along with the experimental conditions used for the conjugation (like the ratio growth-inhibitory agent/antibody, the reaction time, the nature of the solvent and of the cosolvent if any). Thus the contact between the antibody and the growth-inhibitory agent leads to a mixture comprising several conjugates differing from one another by different drug-to-antibody ratios; optionally the naked antibody; optionally aggregates. The DAR that is determined is thus a mean value.

A method which can be used to determine the DAR consists in measuring spectrophotometrically the ratio of the absorbance at of a solution of substantially purified conjugate at λD and 280 nm. 280 nm is a wavelength generally used for measuring protein concentration, such as antibody concentration. The wavelength λD is selected so as to allow discriminating the drug from the antibody, i.e. as readily known to the skilled person, λD is a wavelength at which the drug has a high absorbance and λD is sufficiently remote from 280 nm to avoid substantial overlap in the absorbance peaks of the drug and antibody. λD may be selected as being 252 nm in the case of maytansinoid molecules. A method of DAR calculation may be derived from Antony S. Dimitrov (ed), LLC, 2009, Therapeutic Antibodies and Protocols, vol 525, 445, Springer Science:

The absorbances for the conjugate at λD (AAD) and at 280 nm (A280) are measured either on the monomeric peak of the size exclusion chromatography (SEC) analysis (allowing to calculate the “DAR(SEC)” parameter) or using a classic spectrophotometer apparatus (allowing to calculate the “DAR(UV)” parameter). The absorbances can be expressed as follows:

AλD=(cD×εDλD)+(cA×εAλD)

A280=(cD×εD280)+(cA×εA280)

wherein:
cD and cA are respectively the concentrations in the solution of the drug and of the antibody
εDλD and εD280 are respectively the molar extinction coefficients of the drug at λD and 280 nm
εAλD and εA280 are respectively the molar extinction coefficients of the antibody at λD and 280 nm.

Resolution of these two equations with two unknowns leads to the following equations:

cD=[(εA280×AλD)−(εAλD×A280)]/[(εDλD×εA280)−(εAλD×εD280)]

cA=[A280−(cD×εD280)]/εA280

The average DAR is then calculated from the ratio of the drug concentration to that of the antibody: DAR=cD/cA.

### Cetuximab

The immunoconjugate comprising an antiCEACAM5-antibody is to be used in combination with cetuximab for the treatment of cancer.

Cetuximab (CAS number 205923-56-4) is a chimeric monoclonal IgG1 antibody against epidermal growth factor receptor (EGFR). Cetuximab itself has been used for the treatment of metastatic colorectal cancer, metastatic non-small lung cancer and head and neck cancer.

### Combined Treatment

According to the present invention, the immunoconjugate comprising an anti-CEACAM5-antibody is for use for treating cancer in combination with cetuximab. The invention also relates to cetuximab for use for treating cancer in combination with the immunoconjugate comprising an anti-CEACAM5-antibody.

The present invention also relates to a method of treatment of cancer in a subject in need thereof, comprising administering the immunoconjugate comprising an anti-CEACAM5-antibody, and administering further cetuximab to a subject in need thereof.

The invention also relates to the immunoconjugate comprising an anti-CEACAM5-antibody for use for treating cancer in a subject in need thereof who receives, separately or simultaneously cetuximab.

In an embodiment, the cancer is a solid tumor. According to an embodiment, the cancer is colorectal cancer.

According to an embodiment, the patient is a patient with malignant tumor, in particular with a malignant solid tumor, and more specifically with locally advanced or metastatic solid malignant tumor.

According to an embodiment, the immunoconjugate comprising an anti-CEACAM5-antibody and cetuximab are administered simultaneously to a subject in need thereof.

In a further embodiment, the immunoconjugate comprising an anti-CEACAM5-antibody and cetuximab are formulated (i) in a single pharmaceutical composition comprising the immunoconjugate and cetuximab, or (ii) in the form of two separate pharmaceutical compositions, wherein one pharmaceutical composition comprises the immunoconjugate comprising an anti-CEACAM5-antibody, and the other pharmaceutical composition comprises cetuximab. In the case of formulation of the immunoconjugate and cetuximab in two separate pharmaceutical compositions, the two separate pharmaceutical compositions are administered simultaneously to the subject in need thereof.

According to another embodiment, the immunoconjugate comprising an anti-CEACAM5-antibody and cetuximab are administered separately or sequentially to a subject in need thereof.

According to this embodiment, the immunoconjugate comprising an anti-CEACAM5-antibody and cetuximab are formulated in the form of two separate pharmaceutical compositions, wherein (i) one pharmaceutical composition comprises the immunoconjugate, and (ii) the other pharmaceutical composition comprises cetuximab.

In an embodiment, the immunoconjugate is administered at a dose of from 60 to 210 mg/m². In another embodiment, cetuximab is administered at a dose of from 100 to 600 mg/m².

In another embodiment, the pharmaceutical composition or combination of the present invention is administered, wherein the anti-CEACAM5-antibody is administered at a dose of from 60 to 210 mg and cetuximab is administered at a dose of from 100 to 600 mg. In an aspect of this embodiment, the dosage regimen comprises administration of the dose over a period of 1 h to 48 h, such as over a period of 1 h to 4 h. In an aspect of this embodiment, the dose frequency varies from once a week to once every three weeks. In an embodiment, the treatment duration is of at least 4 or 6 months.

In a further embodiment, the immunoconjugate comprising an anti-CEACAM5-antibody, and cetuximab are administered in 8 to 16 cycles. According to an embodiment, the cycle is selected from a 1-week cycle, a 2-week cycle, or a 3-week cycle. According to an embodiment, one cycle comprises:

administering the immunoconjugate at a dose of from 60 to 210 mg/m²/day, at least once in the cycle;

administering cetuximab at a dose of from 100 to 600 mg/m²/day, at least once in the cycle.

In one embodiment the immunoconjugate is administered at a dose of from 60 to 210 m/m² on day 1 of the cycle. In one embodiment, cetuximab is administered at a dose of from 100 to 600 mg/m² on day 1 and day 5 of the cycle. In one embodiment, cetuximab is administered at a dose of from 100 to 600 mg/m² on day 1 of the cycle.

The unit “mg/m²” indicates the amount of compound in mg/m² of subject body surface administered. The person skilled in the art is aware how to determine the required amount of compound for the subject to be treated based on his body surface, which in turn may be calculated based on height and body weight.

The present invention further relates to a pharmaceutical composition comprising an immunoconjugate comprising an anti-CEACAM5-antibody, and further comprising cetuximab.

The present invention further relates to a kit comprising (i) a pharmaceutical composition comprising the immunoconjugate comprising an anti-CEACAM5-antibody and (ii) a pharmaceutical composition comprising cetuximab, in separate or combined formulations.

The present invention further relates to a pharmaceutical composition comprising an immunoconjugate comprising an anti-CEACAM5-antibody, and further comprising cetuximab for use of treating of cancer.

The present invention further relates to a kit comprising (i) a pharmaceutical composition comprising the immunoconjugate comprising an anti-CEACAM5-antibody and (ii) a pharmaceutical composition comprising cetuximab, in separate or combined formulations, for use for treating of cancer.

“Pharmaceutically” or “pharmaceutically acceptable” refers to molecular entities and compositions that do not produce an adverse, allergic or other untoward reaction when administered to a mammal, especially a human, as appropriate. A pharmaceutically acceptable carrier or excipient refers to a non-toxic solid, semi-solid or liquid filler, diluent, encapsulating material or formulation auxiliary of any type.

As used herein, “pharmaceutically-acceptable carriers” includes any and all solvents, dispersion media, coatings, antibacterial and antifungal agents, and the like that are physiologically compatible. Examples of suitable carriers, diluents and/or excipients include one or more of water, amino acids, saline, phosphate buffered saline, buffer phosphate, acetate, citrate, succinate; amino acids and derivates such as histidine, arginine, glycine, proline, glycylglycine; inorganic salts NaCl, calcium chloride; sugars or polyalcohols such as dextrose, glycerol, ethanol, sucrose, trehalose, mannitol; surfactants such as Polysorbate 80, polysorbate 20, poloxamer 188; and the like, as well as combination thereof. In many cases, it will be preferable to include isotonic agents, such as sugars, polyalcohols, or sodium chloride in the composition, and formulation may also contain an antioxidant such as tryptamine and a stabilizing agent such as Tween 20.

The form of the pharmaceutical compositions, the route of administration, the dosage and the regimen naturally depend upon the condition to be treated, the severity of the illness, the age, weight, and gender of the patient, etc.

The pharmaceutical compositions of the invention can be formulated for a topical, oral, parenteral, intranasal, intravenous, intramuscular, subcutaneous or intraocular administration and the like.

In particular, the pharmaceutical compositions contain vehicles, which are pharmaceutically acceptable for a formulation capable of being injected. These may be in particular isotonic, sterile, saline solutions (monosodium or disodium phosphate, sodium, potassium, calcium or magnesium chloride and the like or mixtures of such salts), or dry, especially freeze-dried compositions which upon addition, depending on the case, of sterilized water or physiological saline, permit the constitution of injectable solutions.

The pharmaceutical composition can be administrated through drug combination devices.

The doses used for the administration can be adapted as a function of various parameters, and in particular as a function of the mode of administration used, of the relevant pathology, or alternatively of the desired duration of treatment.

To prepare pharmaceutical compositions, an effective amount of immunoconjugate comprising an anti-CEACAM5-antibody and of cetuximab may be dissolved or dispersed in a pharmaceutically acceptable carrier or aqueous medium.

The pharmaceutical forms suitable for injectable use include sterile aqueous solutions or dispersions; formulations including sesame oil, peanut oil or aqueous propylene glycol; and sterile powders for the extemporaneous preparation of sterile injectable solutions or dispersions. In all cases, the form must be sterile and injectable with the appropriate device or system for delivery without degradation. It must be stable under the conditions of manufacture and storage and must be preserved against the contaminating action of microorganisms, such as bacteria and fungi.

Solutions of the active compounds as free base or pharmacologically acceptable salts can be prepared in water suitably mixed with a surfactant. Dispersions can also be prepared in glycerol, liquid polyethylene glycols, and mixtures thereof and in oils. Under ordinary conditions of storage and use, these preparations contain a preservative to prevent the growth of microorganisms.

The immunoconjugate comprising an anti-CEACAM5-antibody can be formulated into a composition in a neutral or salt form. Pharmaceutically acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, glycine, histidine, procaine and the like.

The carrier can also be a solvent or dispersion medium containing, for example, water, ethanol, polyol (for example, glycerol, propylene glycol, and liquid polyethylene glycol, and the like), suitable mixtures thereof, and vegetables oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminium monostearate and gelatin.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

The preparation of more, or highly concentrated solutions for direct injection is also contemplated, where the use of DMSO as solvent is envisioned to result in extremely rapid penetration, delivering high concentrations of the active agents to a small tumor area.

Upon formulation, solutions will be administered in a manner compatible with the dosage formulation and in such amount as is therapeutically effective. The formulations are easily administered in a variety of dosage forms, such as the type of injectable solutions described above, but drug release capsules and the like can also be employed.

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, sterile aqueous media which can be employed will be known to those of skill in the art in light of the present disclosure. For example, one dosage could be dissolved in 1 ml of isotonic NaCl solution and either added to 1000 ml of hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the subject being treated. The person responsible for administration will, in any event, determine the appropriate dose for the individual subject.

The immunoconjugate comprising an anti-CEACAM5-antibody formulated for parenteral administration, such as intravenous or intramuscular injection, other pharmaceutically acceptable forms include, e.g. tablets or other solids for oral administration; time release capsules; and any other form currently used.

In certain embodiments, the use of liposomes and/or nanoparticles is contemplated for the introduction of polypeptides into host cells. The formation and use of liposomes and/or nanoparticles are known to those of skill in the art.

Nanocapsules can generally entrap compounds in a stable and reproducible way. To avoid side effects due to intracellular polymeric overloading, such ultrafine particles (sized around 0.1 μm) are generally designed using polymers able to be degraded in vivo. Biodegradable polyalkyl-cyanoacrylate nanoparticles, or biodegradable polylactide or polylactide co glycolide nanoparticules that meet these requirements are contemplated for use in the present invention, and such particles may be are easily made.

Liposomes are formed from phospholipids that are dispersed in an aqueous medium and spontaneously form multilamellar concentric bilayer vesicles (also termed multilamellar vesicles (MLVs)). MLVs generally have diameters of from 25 nm to 4 μm. Sonication of MLVs results in the formation of small unilamellar vesicles (SUVs) with diameters in the range of 200 to 500 Å, containing an aqueous solution in the core. The physical characteristics of liposomes depend on pH, ionic strength and the presence of divalent cations.

### BRIEF DESCRIPTION OF THE SEQUENCES

SEQ ID NO: 1-5 show the sequences CDR1-H, CDR2-H, CDR3-H, CDR1-L and CDR3-L of the anti-CEACAM5-antibody (huMAb2-3).

SEQ ID NO: 6 shows the sequence of the variable domain of the heavy chain (VH) of the anti-CEACAM5-antibody (huMAb2-3).

SEQ ID NO: 7 shows the sequence of the variable domain of the light chain (VL) of the anti-CEACAM5-antibody (huMAb2-3).

SEQ ID NO: 8 shows the heavy chain sequence of the anti-CEACAM5-antibody (huMAb2-3).

SEQ ID NO: 9 shows the light chain sequence of the anti-CEACAM5-antibody (huMAb2-3).

### BRIEF DESCRIPTION OF THE FIGURES

FIG. 1: Activity of immunoconjugate huMAb2-3-SPDB-DM4 and cetuximab as single agents or in combination against subcutaneous colon patient-derived xenograft (PDX) CR-IGR-0007P PDX in SCID mice. Tumor volume evolution by treatment group. The curves represent medians+ or − MAD (Median Absolute Deviation) at each day for each group.

FIG. 2: Activity of immunoconjugate huMAb2-3-SPDB-DM4 and cetuximab as single agents or in combination against subcutaneous colon patient-derived xenograft CR-IGR-0011C PDX, in SCID mice. Tumor volume evolution by treatment group. The curves represent medians+ or −MAD at each day for each group.

### EXAMPLES

### Example 1: Activity of Immunoconjugate huMAb2-3-SPDB-DM4 in Combination with Cetuximab Against Two Subcutaneous Colon Patient-Derived Xenografts CR-IGR-0007P PDX and CR-IGR-0011C PDX in SCID Mice

Experimental Procedure

The activity of huMAb2-3-SPDB-DM4 and cetuximab regimen was evaluated as single agent or in combination in two subcutaneous colon patient-derived xenografts (PDX) (CR-IGR-0007P PDX and CR-IGR-0011C PDX) implanted s.c. in female SCID mice. Control groups were left untreated. The doses of the compounds used are given in mg/kg.

For the CR-IGR-0007P PDX, treatments were initiated on day 26 post tumour implantation when median tumour burden reached 166.0 mm³. huMAb2-3-SPDB-DM4 was administered at 5 mg/kg following 3 weekly cycles of IV administrations on days 26, 33 and 40. Cetuximab was administered at 10 mg/day as IV administrations on days 26, 33, 40, and 44.

For the CR-IGR-0011C PDX, treatments were initiated on day 19 post tumour implantation when median tumour burden reached 123.5 mm³. huMAb2-3-SPDB-DM4 was administered at 5 mg/kg following 3 weekly cycles of IV administrations on days 19, 26 and 33. Cetuximab was administered at 10 mg/kg as IV administrations on days 19, 23, 26, 30 and 33.

For the evaluation of anti-tumor activity, animals were weighed daily and tumors were measured 2 times weekly by caliper. A dosage producing a 20% weight loss at nadir (mean of group) or 10% or more drug deaths, was considered an excessively toxic dosage. Animal body weights included the tumor weights. Tumor volume were calculated using the formula mass (mm³)=[length (mm)×width (mm)×width (mm)]/2. The primary efficacy end points are ΔT/ΔC, percent median regression, partial and complete regressions (PR and CR).

Changes in tumor volume for each treated (T) and control (C) are calculated for each tumor by subtracting the tumor volume on the day of first treatment (staging day) from the tumor volume on the specified observation day. The median ΔT is calculated for the treated group and the median ΔC is calculated for the control group. Then the ratio ΔT/ΔC is calculated and expressed as a percentage: ΔT/ΔC=(delta T/delta C)×100.

The dose is considered as therapeutically active when ΔT/ΔC is lower than 40% and very active when ΔT/ΔC is lower than 10%. If ΔT/ΔC is lower than 0, the dose is considered as highly active and the percentage of regression is dated (Plowman J, Dykes D J, Hollingshead M, Simpson-Herren L and Alley MC. Human tumor xenograft models in NCI drug development. In: Feibig HH BA, editor. Basel: Karger.; 1999 p 101-125):

% tumor regression is defined as the % of tumor volume decrease in the treated group at a specified observation day compared to its volume on the first day of first treatment.

At a specific time point and for each animal, % regression is calculated. The median % regression is then calculated for the group:

$$\% regression ( at t ) = \frac{{volume}_{t 0} - {volume}_{t}}{{volume}_{t 0}} \times 100$$

Partial regression (PR): Regressions are defined as partial if the tumor volume decreases to 50% of the tumor volume at the start of treatment.

Complete regression (CR): Complete regression is achieved when tumor volume=0 mm³ (CR is considered when tumor volume cannot be recorded).

Results

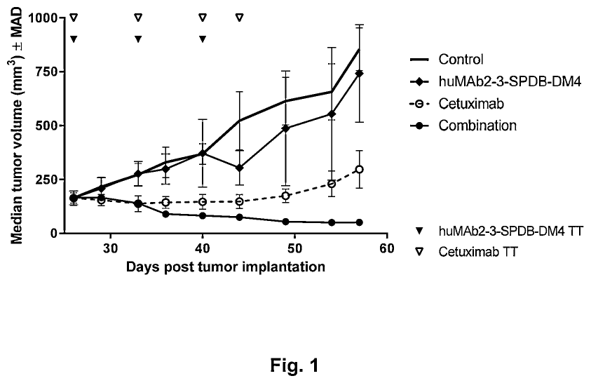

The results for the CR-IGR-0007P PDX are presented on FIG. 1 and Table 1 (below).

One mouse of control group was found dead on D54; the CR-IGR-0007P is an aggressive tumor and can be cachexic. huMAb2-3-SPDB-DM4 and cetuximab were administered at doses lower than maximal tolerated dose (MTD) and treatments were well tolerated and did not induce toxicity.

The huMAb2-3-SPDB-DM4 as a single agent was inactive with a ΔT/ΔC on D49 equal to 76%. The cetuximab regimen as single agent was very active with a ΔT/ΔC equal to 2% (p<0.0001).

The combined huMAb2-3-SPDB-DM4 and cetuximab regimen was ΔT/ΔC inferior to 0% (p<0.0001), a tumor regression of 67% and 6 PR (partial regression). The effect of the combination of huMAb2-3-SPDB-DM4 with cetuximab was significantly different from the effect of cetuximab alone from day 36 to day 62 and significantly different from the effect of huMAb2-3-SPDB-DM4 alone from day 33 to 62.

In conclusion in the CR-IGR-0007P PDX, huMAb2-3-SPDB-DM4 after 3 weekly IV administrations at 5 mg/kg was inactive as single agent. Cetuximab regimen was very active and the treatment was well tolerated. The combination of the huMAb2-3-SPDB-DM4 and cetuximab regimen was more active than the single agents.

TABLE 1
Activity of huMAb2-3-SPDB-DM4 and cetuximab in combination against subcutaneous colon
Patient-Derived-Xenograft, CR-IGR-0007P in SCID mice

| | | Dosage in | | | | | | | | | |
|---|---|---|---|---|---|---|---|---|---|---|---|
| | Route | mg/kg | | Drug | Mean body | | Median | | | | |
| | (Dosage | (total | | death | weight change | Median | % of | | | Biosatitic | |
| | in mL/ | cumulated | Schedule in | (day of | in % at nadir | ΔT/ΔC in | regression | Regression | | p value^{a} | Biological |
| Agent | kg) | dose) | day | death) | (day of nadir) | % (D49) | (D49) | PR | CR | (D49) | comments |
| Cetuximab | IV (10) | 10 (40) | 26, 33, 40, 44 | 0/6 | −4.2 (45) | 2 | — | 0/6 | 0/6 | <0.0001 | Very active |
| huMAb2-3- | IV (10) | 5 (15) | 26, 33, 40 | 0/6 | −3.4 (54) | 76 | — | 0/6 | 0/6 | 0.1068 | Inactive |
| SPDB-DM4 | | | | | | | | | | | |
| Cetuximab | IV (10) | 10 (40) | 26, 33 ,40, 44 | 0/6 | −4.7 (40) | <0 | 67 | 6/6 | 0/6 | <0.0001 | Very active |
| huMAb2-3- | IV (10) | 5 (15) | 26, 33, 40 | | | | | | | | |
| SPDB-DM4 | | | | | | | | | | | |
| Control | — | — | — | 0/6 | −7.0 (57) | — | — | — | — | — | — |

^{a}Statistical analysis. The p-values were obtained using a contrast analysis to compare each treated group versus control using Bonferroni-Holm adjustment for multiplicity after a two-way Anova-Type with repeated measures on tumor volume changes from baseline. A probability less than 5% (p < 0.05) was considered as significant.
ΔT/ΔC = ratio of medians of tumor volume changes from baseline between treated and control groups;
PR = Partial regression;
CR = Complete regression

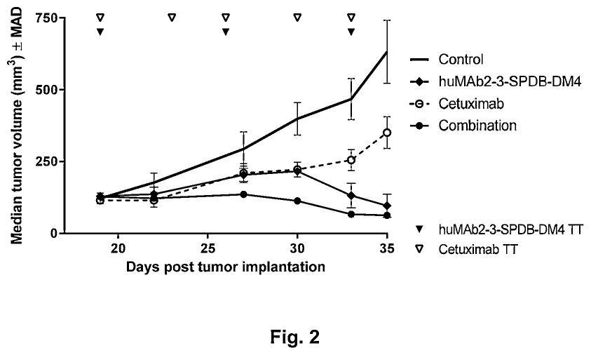

The results for the CR-IGR-0011C PDX are presented on FIG. 2 and Table 2 (below).

Mice of control group exhibited negative body weight changes (nadir of −6.7% on Day 32); the CR-IGR-0011C is an aggressive tumor and can be cachexic. huMAb2-3-SPDB-DM4 and cetuximab were administered at doses lower than maximal tolerated dose (MTD) and treatments were well tolerated and did not induce toxicity.

The huMAb2-3-SPDB-DM4 as single agent was highly active with a ΔT/ΔC on D35 inferior to 0% (p<0.0001), a tumor regression of 29% and 2 PR (partial regression). The cetuximab regimen as single agent inactive with a ΔT/ΔC equal to 44% (NS).

The combination of huMAb2-3-SPDB-DM4 and cetuximab regimen was highly active with a ΔT/ΔC inferior to 0% (p<0.0001), a tumor regression of 52% and 3 PR. The effect of the combination of huMAb2-3-SPDB-DM4 with cetuximab was significantly different from the effect of huMAb2-3-SPDB-DM4 alone from day 30 to day 33 and significantly different from the effect of cetuximab alone from day 30 to 35.

In conclusion, in the CR-IGR-0001C PDX, huMAb2-3-SPDB-DM4 after 3 weekly IV administrations at 5 mg/kg was highly active as single agent. Cetuximab was inactive as single agent. The combination of HUMAB2-3-SPDB-DM4 with cetuximab was significantly more active than the single agents.

TABLE 2
Activity of HUMAB2-3-SPDB-DM4 and cetuximab in combination against subcutaneous
colon Patient-Derived-Xenograft, CR-IGR-0011C in SCID mice

| | | Dosage in | | | | | | | | | |
|---|---|---|---|---|---|---|---|---|---|---|---|
| | Route | mg/kg | | Drug | Mean body | | Median | | | | |
| | (Dosage | (total | | death | weight change | Median | % of | | | Biosatitic | |
| | in mL/ | cumulated | Schedule in | (day of | in % at nadir | ΔT/ΔC in | regression | Regression | | p value^{a} | Biological |
| Agent | kg) | dose) | day | death) | (day of nadir) | % (D35) | (D35) | PR | CR | (D35) | comments |
| Cetuximab | IV (10) | 10 (50) | 19, 23, 26, 30, 33 | 0/6 | −3.9 (33) | 44 | — | 0/6 | 0/6 | 0.1528 | Inactive |
| HUMAB2-3- | IV (10) | 5 (15) | 19, 26, 33 | 0/6 | −6.2 (25) | <0 | 29 | 2/6 | 0/6 | <0.0001 | Highly |
| SPDB-DM4 | | | | | | | | | | | active |
| Cetuximab | IV (10) | 10 (40) | 19, 23, 26, 30, 33 | 0/6 | −2.9 (24) | <0 | 52 | 3/6 | 0/6 | <0.0001 | Highly |
| HUMAB2-3- | IV (10) | 5 (15) | 19, 26, 33 | | | | | | | | active |
| SPDB-DM4 | | | | | | | | | | | |
| Control | — | — | — | 0/6 | −6.7 (32) | — | — | — | — | — | — |

^{a}Statistical analysis. The p-values were obtained using a contrast analysis to compare each treated group versus control using Bonferroni-Holm adjustment for multiplicity after a two-way Anova-Type with repeated measures on tumor volume changes from baseline. A probability less than 5% (p < 0.05) was considered as significant.
ΔT/ΔC = ratio of medians of tumor volume changes from baseline between treated and control groups;
PR = Partial regression;
CR = Complete regression

1. An immunoconjugate comprising an anti-CEACAM5-antibody for use for treating cancer in combination with cetuximab.

2. The immunoconjugate for the use of claim 1, wherein the anti-CEACAM5-antibody comprises a CDR-H1 consisting of SEQ ID NO: 1, CDR-H2 consisting of SEQ ID NO: 2, CDR-H3 consisting of SEQ ID NO: 3, CDR-L1 consisting of SEQ ID NO: 4, CDR-L2 consisting of amino acid sequence NTR, and CDR-L3 consisting of SEQ ID NO: 5.

3. The immunoconjugate for the use of claim 1 or 2, wherein the anti-CEACAM5-antibody comprises a variable domain of a heavy chain (VH) consisting of SEQ ID NO: 6 and a variable domain of a light chain (VL) consisting of SEQ ID NO: 7.

4. The immunoconjugate for the use of any of claims 1 to 3, wherein the anti-CEACAM5-antibody comprises a heavy chain (VH) consisting of SEQ ID NO: 8 and a light chain (VL) consisting of SEQ ID NO: 9.

5. The immunoconjugate for the use of any of claims 1 to 4, wherein the immunoconjugate comprises at least one cytostatic agent.

6. The immunoconjugate for the use of claim 5, wherein the cytostatic agent is selected from the group consisting of radioisotopes, protein toxins, small molecule toxins, and combinations thereof.

7. The immunoconjugate for the use of claim 6, wherein the small molecule toxins are selected from antimetabolites, DNA-alkylating agents, DNA-cross-linking agents, DNA-intercalating agents, anti-microtubule agents, topoisomerase inhibitors, and combinations thereof.

8. The immunoconjugate for the use of claim 7, wherein the anti-microtubule agent is selected from the group consisting of taxanes, vinca alkaloids, maytansinoids, colchicine, podophyllotoxin, gruseofulvin, and combinations thereof.

9. The immunoconjugate for the use of claim 8, wherein the maytansinoids are selected from the group consisting of N2′-deacetyl-N2′-(3-mercapto-1-oxopropyl)-maytansine (DM1) or N2′-deacetyl-N-2′(4-methyl-4-mercapto-1-oxopentyl)-maytansine (DM4), and combinations thereof.

10. The immunoconjugate for the use of any of claims 1 to 9, wherein the anti-CEACAM5-antibody is covalently attached via a cleavable or non-cleavable linker to the at least one cytotoxic agent.

11. The immunoconjugate for the use of claim 10, wherein said linker is selected from the group consisting of N-succinimidyl pyridyldithiobutyrate (SPDB), 4-(pyridin-2-yldisulfanyl)-2-sulfo-butyric acid (sulfo-SPDB), and succinimidyl(N-maleimidomethyl) cyclohexane-1-carboxylate (SMCC).

12. The immunoconjugate for the use of any of claims 1 to 11, comprising an CEACAM5-antibody, which comprises a heavy chain (VH) consisting of SEQ ID NO: 8 and a light chain (VL) consisting of SEQ ID NO: 9 (huMAb2-3), and which is covalently linked to N2′-deacetyl-N-2′(4-methyl-4-mercapto-1-oxopentyl)-maytansine (DM4) via N-succinimidyl pyridyldithiobutyrate (SPDB).

13. The immunoconjugate for the use of any of claims 1 to 12, wherein the immunoconjugate is characterised by a drug-to-antibody ratio (DAR) ranging from 1 to 10.

14. The immunoconjugate for the use of any of claims 1 to 13, wherein the cancer is colorectal cancer.

15. The immunoconjugate for the use of any of claims 1 to 14, wherein the immunoconjugate and cetuximab are administered simultaneously to a subject in need thereof.

16. The immunoconjugate for the use of claim 15, wherein the immunoconjugate and cetuximab are formulated (i) in a single pharmaceutical composition comprising the immunoconjugate and cetuximab, or (ii) in the form of two separate pharmaceutical compositions, wherein one pharmaceutical composition comprises the immunoconjugate, and on the other pharmaceutical composition comprises cetuximab.

17. The immunoconjugate for the use of any of claims 1 to 14, wherein the immunoconjugate and cetuximab are administered separately or sequentially to a subject in need thereof.

18. The immunoconjugate for the use of claim 17, wherein the immunoconjugate and cetuximab are formulated in the form of two separate pharmaceutical compositions, wherein (i) one pharmaceutical composition comprises the immunoconjugate, and (ii) the other pharmaceutical composition comprises cetuximab.

19. The immunoconjugate for the use of any of claims 1 to 18, wherein the immunoconjugate comprising an anti-CEACAM5-antibody, and cetuximab are administered in 8 to 16 cycles wherein one cycle comprises:
administering the immunoconjugate at a dosage of from 60 to 210 mg/m², at least once in a cycle;
administering cetuximab at a dosage of from 100 to 600 mg/m² at least once in a cycle.

20. A pharmaceutical composition comprising the immunoconjugate of any of claims 1 to 14, and cetuximab.

21. A kit comprising (i) a pharmaceutical composition of the immunoconjugate of any of claims 1 to 14 and (ii) a pharmaceutical composition comprising cetuximab.

22. The pharmaceutical composition according to claim 20 or the kit according to claim 21 for the use for treating cancer.
